(19) United States
(12) Patent Application Publication
(10) Pub. No.: US 2004/0241119 A1
(43) Pub. Date: Dec. 2, 2004

(54) Cleo 2

(76) Inventor: Leonard Bell Curry, Suitland, MD (US)

Correspondence Address: LEONARD BELL CURRY, 4620 Lacy Avenue, SUITLAND, MD, 20746-1504, US

(21) Appl. No.: 10/444,871

(22) Filed: May 27, 2003

(51) Int. Cl. A61K 7/025

(52) U.S. Cl. 424/64

(57) ABSTRACT

This idea of new invention is to simply add artificial and or natural flavorings being any and all the flavors the World, to all of the Lipsticks there are past, present and or future makings they're of. Being any types, styles, shades and colors. If I am allowed to that is.

### RELATED APPLICATION

N/A

### FEDERALLY SPONSORED

N/Y

### MICROFICHE APPENDIX

N/Y

Background of the Invention is all Lipsticks of every shades, colors, types and styles. Brief Summary of the Invention is to simply add any and all the flavors of the world into Lipsticks, any and or, all.

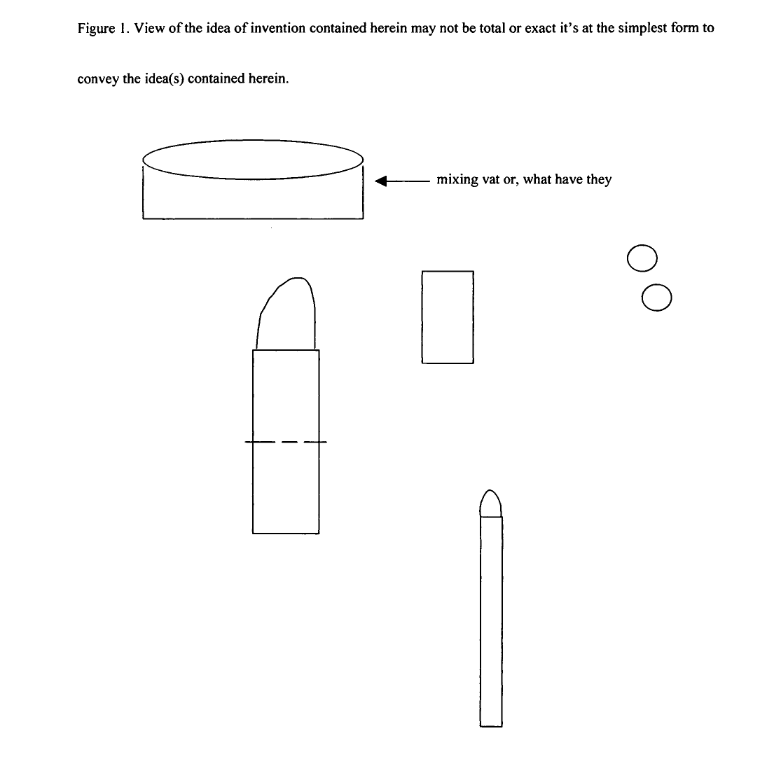

The View. FIG. 1. View of the idea

Detailed Description of the Invention pertains to make in any country in a factory(s) or what have they, new flavored Lipsticks. These new lipsticks will be better then the old because of the new added flavors. These new flavored Lipsticks will be flavored artificially and or, naturally. This new flavoring can be added to any and all Lipsticks of any and all shades, styles, colors and types of yesterdays, today's and tomorrows Lipsticks. For instance, the new flavors will be anything from fruits such as watermelon, to candy and ice-cream. Any and all flavors of the world can be used to flavor these new flavored Lipsticks. These new flavored Lipsticks can be made as all lipsticks are made past, present, future. The modification if any, that might be needed, if it's done automatically, can be done to machinery in regards to adding flavor into the lipstick. Sequence Listing: Lipsticks any and or all. Artificial and or natural flavoring.

1. I Leonard Bell Curry do hereby make claim domestic as well as foreign to the new idea for invention contained herein which is to add artificial and or natural flavoring any and all flavors of the world of the past, present and future flavorings to any and or all Lipstick style, types and shades, colors of the past, present and future if wanted, for marketing. And I would imagine any machinery modifications and or, improvements there of that would be needed to make this new idea obtainable if wanted.
